(19) United States
(12) Patent Application Publication
(10) Pub. No.: US 2023/0060576 A1
(43) Pub. Date: Mar. 2, 2023

(54) Laparoscopic Surgeon's Knot Maker

(71) Applicant: Peter Fan, Englewood Cliffs, NJ (US)

(72) Inventor: Peter Fan, Englewood Cliffs, NJ (US)

(21) Appl. No.: 17/410,747

(22) Filed: Aug. 24, 2021

(51) Int. Cl. A61B 17/04

(52) U.S. Cl. CPC A61B 17/0485; A61B 17/0482; A61B 17/0469

(57) ABSTRACT

Tying knots in a suture laparoscopically with straight graspers is difficult, time consuming, inefficient, and ineffective. Many instruments have been invented for this purpose, but the common problem that leads to failure is that the first throw in the tie tends to unravel itself before the second throw can be carried out. The current invention consisting of 3 mini-graspers within the same shaft, is capable of quickly producing two loops in the first throw, which is self-locking, leading to the formation of a Surgeon's Knot.

### FIELD OF INVENTION

The Davinci Machine can easily tie knots intra-abdominally, but it is very expensive, not widely available and difficult to learn. The average laparoscopic surgeon in the absence of the Davinci, still needs an instrument that helps in the manual intra-corporeal tying of knots. A major problem with tying of knots is that the initial single loop tie tends to unravel itself before the next throw is thrown, which is the reason why surgeons in open surgery developed the “Surgeon's Knot” using 2 loops in the first throw. The present invention can make these 2 loops quickly and easily laparoscopically.

The prior art in “The Laparoscopic Loop Maker”, U.S. Pat. No. 9,820,736, by this author, enabled the formation a loop in the head end of the suture, followed by pulling the tail end of the suture through this loop. However, this produced only one loop, and additional loops required repeated maneuvers. The problem with one loop ties is that after first throw the knot tended to become loose before the second throw was thrown. With the surgeon's knot which uses 2 loops in the first throw, the advantage is that after the first throw the loops will self-lock, and will not loosen up by themselves. It is therefore the object of the present invention to enable the making of the “Surgeon's Knot” laparoscopically.

### BACKGROUND OF THE INVENTION

Tying of knots is essential in any kind of surgery. It is relatively easy in open surgery but difficult in laparoscopic surgery. This is because the surgeon must use two laparoscopic graspers, one in each hand, with both graspers trapped in the wall of the abdomen. The current art of laparoscopic knot tying employs either the extra-corporeal method, involving tying the knot by hand outside the body and pushing the knot inside with a knot pusher, or the intra-corporeal method, involving manipulation of the suture with the tips of two laparoscopic graspers, which is slow and cumbersome, and requires considerable skill. Laparoscopic clip appliers, staplers, pre-tied knots and the like are useful substitutes, but cannot totally replace tied knots which are still needed. Despite considerable prior art, today hardly any hand operated instrument exists that renders laparoscopic intra-corporeal knot tying easier and faster.

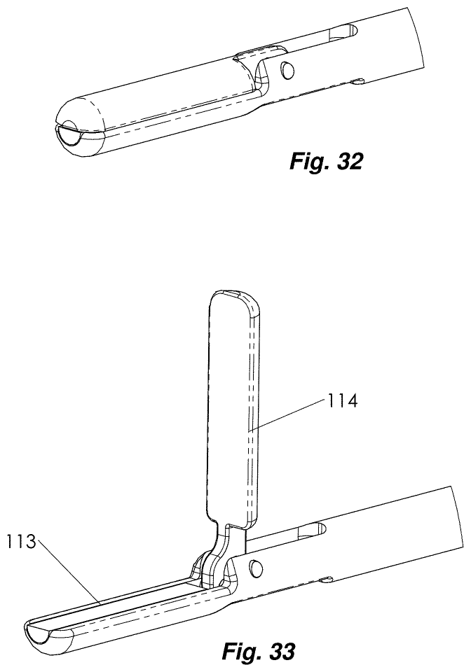

In order to describe the tying process, the different parts of a suture ligature are first given names. As shown in FIG. 32 and FIG. 33 in the drawings, after the suture ligature has passed around the tissue to be tied, it then presents with a head end 144, a head strand 145, a tail strand 146, and a tail end 147.

There are three basic methods of tying a knot, whether done openly or laparoscopically. The first method makes the head end of the suture pass 360 degrees continuously around the tail strand, as is shown in the U.S. Pat. No. 9,561,028, “Automatic Laparoscopic Knot Tier”, invented by this author, and was designed specifically to avoid the release and re-grab. In practice, this instrument failed because in the final take-away, the head end of the suture was held by a miniature compression spring, which is unreliable.

The second method makes a loop, which is commonly used by surgeons performing open surgery, where the surgeon makes “instrument ties”, by wrapping the tail strand of the suture around the needle holder, and then pulling the head end of the suture through the loop. In the open instrument tie, the surgeon could make one loop or several loops with the needle-holder. A method of making a loop laparoscopically was described in the U.S. Pat. No. 9,820,736, 2017, by this author. However, this made only one loop, and additional loops required repeat maneuvers.

The third method involves making a “throw”, which actively passes the head end of the suture behind its tail strand, between two adjacent graspers. This simulates the tying of shoe-laces by fingers and requires the release and re-grab of the head end of the suture, behind the tail strand, which was described in U.S. Pat. No. 10,433,835, 2019, and U.S. Pat. No. 10,405,852, 2019, also by this author. In practice this also failed because the final take-away also depended on springs, and the method was too complicated.

The previous patent of “The Laparoscopic Suture Loop Maker”, U.S. Pat. No. 9,820,736, Nov. 21, 2017, also by this author, described an effective method of producing a loop in the suture, by grasping the suture simultaneously at two different points, and using a sliding mechanism, but it did not address the problems in the next step of the tying process. It failed to tie the knot because (1) the stationary grasper it used, depended solely on a compression spring to hold the head end of the suture in the final take-away, which was inadequate, as the suture always slipped off, (2) the straight in-line handle it used was like the handle of a racket, so neither the scissors grip nor the pistol grip could be added or used. In a follow-up improvement, the straight in-line handle was replaced by the scissor-handles of the regular grasper, as well as by a pistol grip for the sliding grasper, but this instrument also produced only a single loop.

A recent invention by this author, the “Laparoscopic Loop Maker with Rotating Mini-Grasper”, U.S. Pat. No. 17,321,467, August 2021, uses a unique rotating sheath to make multiple loops as required in the “Surgeon's Knot”. The rotating sheath is not part of a grasper, but slides over the shaft of a standard 5 mm grasper, and the two are used together. One end of the suture is trapped by a unique side-opening single-acting jaw of a mini-grasper installed within the walls of the sheath. This enabled loops in the suture to wind around the outside of the sheath, simply by rotating the sheath. One could make as many loops as one desired, but the instrument had drawbacks. First it involves using a separate regular grasper to grab the head end of the suture, and pass it to the rotating mini-grasper, and later on, it involves pushing the loops of the suture off the shaft of the rotating instrument, and re-grabbing of the head end of the suture by the separate regular grasper. The current invention avoids these un-necessary maneuvers.

### General Discussion on Other Similar Past Patents

There have been many devices that helped to “create a loop”, with each functioning differently—(a) Kitano's grasper with the rotating sleeve, Japanese, (b) Donald Murphy's grasper with the extra horn, Australian, (c) Grice's sleeve catching instrument, (d) Bagnato & Wilson's device which simulates the radiological pig-tail catheter, with a preformed loop built into the tip of the catheter, which is deformable and purportedly a loop former, but it is difficult to manufacture and apply, and has not yet been reduced to practice, (e) Ferzli's double grasper, which anchors one end of the suture, as described above. There have also been devices using a “pre-formed knot”, (1) Ethicon's Endo-Loop, (2) the Duraknot, (3) LSI's device, (4) Pare's pre-tied knot, all of which do not help to make loops or knots.

Other past inventions related to intra-corporeal laparoscopic knot tying failed to address the basic problem of “how to create a loop”. They usually offer various alternatives, such as making fishing knots, using pre-tied knots, knot pushers, suture clips, cinchers, tissue fasteners, anchors, stapling devices, etc. The present invention however can actively makes two loops in the head end of the suture, and allows the tail end of the suture to pass through the middle of the loops, to make the knot.

### Detailed Discussion on Other Similar Past Patents

The Ferzli instrument, 1993, U.S. Pat. No. 5,201,759 —Combined the shafts of two graspers into one, so the cross section of the combined shaft was oval, not round. The two graspers shared the same stationary handle, so the instrument had a total of 3 classical finger-loop handles. The intent was for two pairs of jaws to grasp the same suture at two different points, which made that portion of the suture steady, and helped in making a loop, but there is no rotation of anything. This not been reduced to practice. “The Medical Knot Tying Instrument” of Drew Grice, U.S. Pat. No. 5,437,682, Aug. 1, 1995—describes an instrument where the gripping mechanism is the divided sheath, with the outer sheath divided into 3 portions, providing 2 gripping areas, which allowed the loops to be made by rotation, but the gripping method is uncertain.

The Kitano Grasper, 1996, Minimal Invasive Therapy & Allied Technology, Vol 5, 27 & 28.—Mentions the use of a “side grasper”, in which the grasper's sheath is divided into parts, with the suture caught between the circular ends of an upper sheath and a lower sheath. Rotation of the entire grasper would result in a loop being formed. This catching mechanism is similar to Grice's, is time consuming, is not reliable, and is not in use.

The Fan's Laparoscopic Loop Maker, U.S. Pat. No. 9,820,736, has two parallel graspers side by side within the same outer sheath, with one grasper being able to slide out, grasping the same suture at a second point. Pulling back the sliding grasper produced a loop, helping to make the knot, but it formed only one loop. Additional loops required additional maneuvers. The problem was the first throw with the single loop tended to unravel itself.

The Ratnapala instrument, Patent 2019/0239870, consists of one grasper whose shaft is surrounded by an outer rotating tube having a small side-slit for trapping one end of the suture. It is difficult to see how the jaws work, since they do not have the usual handles. Rotating the outer tube is supposed to produce loops around the shaft, but the mechanism is undefined. The principle of rotating the outer tube or sheath was already shown in Grice 1995 and Kitano 1996. The side-slit as a catching mechanism is time-consuming and difficult to use and is not reliable.

The Fan's Laparoscopic Knot Tying Instrument, U.S. Pat. No. 17,000,332, is similar to U.S. Pat. No. 9,820,736, with an improved handle, but also produced only one loop.

The Fan's Push-twist suture-passing laparoscopic knot tying instrument, U.S. Pat. No. 10,405,852 has two parallel graspers, and produces a knot by passing the tail end of the suture from one grasper to the other. This produced only one loop.

“Surgical Instrument for Forming a Knot”—Schulze et. al.—December 1998—U.S. Pat. No. 5,846,254, describes a complicated and cumbersome method of forming loops for knot tying.

U.S. PAT. DOCUMENTS

| | | | | |
|---|---|---|---|---|
| 1. | 3,834,395 | Sep. 10, 1974 | Manuel Santos | 128/326 |
| 2. | 5,201,759 | Apr. 13, 1993 | George Ferzli. | 606/139 |
| 3. | 5,217,471 | Jun. 08, 1993 | Stephen Burkhart | 606/148 |
| 4. | 5,234,443 | Aug. 10, 1993 | Phan & Stoller | 606/148 |
| 5. | 5,250,054 | Oct. 5, 1993 | Lehmann Li | 606/148 |
| 6. | 5,281,236 | Jan. 25, 1994 | Bagnato et al. | 606/139 |
| 7. | 5,312,423 | May 17, 1994 | Rosenbluth & Brenneman | 606/148 |
| 8. | 5,395,382 | Mar. 7, 1995 | DiGiovanni et al. | 606/148 |
| 9. | 5,437,682 | Aug. 1, 1995 | Drew Grice | 606/148 |
| 10. | 5,423,836 | Jun. 13, 1995 | Scott Brown | 606/148 |
| 11. | 5,439,467 | Aug. 8, 1995 | Theodore Benderev, et al. | 606/139 |
| 12. | 5,480,406 | Jan. 2, 1996 | Nolan et al. | 606/139 |
| 13. | 5,728,109 | Mar. 17, 1998 | Schulze et al. | 606/148 |
| 14. | 5,810,852 | Sep. 22, 1998 | Greenberg et al. | 606/148 |
| 15. | 5,814,054 | Sep. 29, 1998 | Kortenbach et al. | 606/139 |
| 16. | 5,846,254 | Dec. 8, 1998 | Schulze et al. | 606/228 |
| 17. | 6,051,006 | Apr. 18, 2000 | Shluzas & Sikora | 606/148 |
| 18. | 6,086,601 | Jul. 1, 2000 | InBae Yoon | 606/148 |
| 19. | 6,221,084 | Apr. 24, 2001 | R. Fleenor, Pare Surgical | 606/148 |
| 20. | 6,432,118 | Aug. 13, 2002 | Mollenhauer & Kucklick | 606/148 |
| 21. | 6,716,224 | Apr. 26, 2004 | Singhatat | 606/148 |
| 22. | 2009/0228025 | Sep. 10, 2009 | Steven Benson | 606/144 |
| 23. | 2010/0016883 | Jan. 21, 2010 | George Christoudias | 606/205 |
| 23. | 5,312,423 | May 17, 1994 | Rosenbluth et al. | 606/148 |
| 25 | 8,512,362 | Aug. 20, 2013 | Ewers et al. | 606/158 |
| 26. | 4,635,638 | Jan. 1987 | Weintraub. | |
| 27. | 5,938,668 | Aug. 1999 | Scirica. | |
| 28. | 5,954,731 | Sep. 1999 | Yoon. | |
| 29. | 6,017,358 | Jan. 2000 | Yoon | |
| 30. | 6,086601 | Jul. 2000 | Yoon. | |
| 31. | 2008/0228204 | Sep. 2008 | Hamilton. | |

| | | | |
|---|---|---|---|
| 32. | 2013/051992 | Sep. 30, 2012 | Fan - Double Laparoscopic Grasper. |
| 33. | 9,561,028 | Feb. 7, 2017 | Fan - Automatic Lap. Knot |
| | | | Instrument |
| 34. | 9,820,736 | Nov. 21, 2017 | Fan - Laparoscopic Suture Loop |
| | | | Maker. |
| 35. | 10,433,835 | Oct. 08, 2019 | Fan - Suture Passing Laparoscopic |
| | | | Knot-tier. |
| 36. | 10,405,852 | Sep. 10, 2019 | Fan - Push-Twist Suture-Passing |
| | | | Laparoscopic Knot Tying |
| | | | Instrument. |
| 37. | 2019/0239870 | | The Ratnapala Instrument |
| 38. | 2021/17321467 | Aug. 05, 2021 | Fan - Laparoscopic Loop Maker |
| | | | with Rotating Mini-Grasper. |

### Other Publications

1. Endo-stitch—Autosuture—Manufacturer's item #173016.
2. Maniceps—Japanese suturing device, similar to Endo-stitch.
3. A Laparoscopic Device for Minimally Invasive Cardiac Surg. Shaphan Jernigan, et. al.—European J. of Cardio-thoracic Surgery, Vol. 37, p.626-630. March 2010.
4. Knot Tying Intra-corporeally, with newly designed Forceps. (sliding sleeve). Kitano et. al.—J. of Minimal Invasive Therapy & Allied Tech, 1996. 5: 27-28.
5. Endoscopic Knot Tying Made Easier—(one jaw with extra bump).
6. Donald Murphy—ANZ J. Surg. 1995. 65, 507-509.
7. The Excalibur Suturing Needle Holder—(jaw with prominent heel, helps looping)
8. Uchida et. al. Surgical Endoscopy—vol. 3, 531-532
9. Alijizawi laparoscopic auto-knot device—(two dissolving balls).
10. A New Reusable Instrument designed for simple and secure knot tying in laparoscopic surgery. S. S. Miller 1996 Surg. Endos 10: 940-941(pointed canula).
11. The Nobel Automatic Laparoscopic Knotting and Suturing Device. Mishra et. al. World Laparoscopy Hospital, India. (a knot pusher)
12. Automated Knot Tying for Fixation in Minimally Invasive Robot Assisted Cardiac Surgery. March 9(1):105-12.
13. Kuniholm & Buckner—J. Biomed Engino 2005, Vol.127,1001-8. JSLS.2005 January
14. MI Frecke—Laparoscopic multifunctional instruments: design and testing. Endosc Surg Allied Technol. 1994 December; 2(6):318-9.
15. G. Berci—Multifunctional laparoscopic Instruments.
16. http://www.ligasure.com/ligasure/pages.aspx?page=Products/Laparoscopi
17. http://www.freepatentsonline.com/y2010/0063437
18. http//www.ncbi.nlm.nih.gov/pubmed/15791983 Multifunctional Laparoscopic Instruments.

### SUGGESTED U.S. CLASSIFICATION: 606/139, 144, 145,148.

### SUGGESTED INTERNATIONAL CLASSIFICATION: A61B 17/00, 04, 28.

### FIELD OF SEARCH: 606/139, 144, 145, 147, 148, 150, 151, 127,128, 606/167, 168, 170, 174, 182, 185, 205, 207, 210, 211.

RELATED PRIOR PATENTS: 2013/051,992 U.S. Pat. No. 9,194,468. U.S. Pat. No. 9,561,028. U.S. Pat. No. 9,820,736. U.S. Pat. No. 10,405,852. and 10,433,835.

### SUMMARY OF THE INVENTION:

In laparoscopic surgery, the tying of knots intra-corporeally is typically performed manually using the tips of two laparoscopic graspers. It is still technically difficult and requires considerable skill and practice. The advent of laparoscopic clips and staples was a great blessing to surgeons, but could not totally replace the use of tied knots, which is still necessary. The laparoscopic instrument presented here basically consists of 3 mini-graspers, which are used to create 2 loops in the suture, necessary for the first throw in the surgeon's knot.

NUMBERS AND NAMES OF PARTS -

```
  1. Main Sheath.
  2. Front Spacer.
  3. Rear Spacer.
  4. Chamber with Handle.
  5. Slidable Mini-Grasper-A
  6. Slidable Mini-Grasper-B
  7. Stationary Mini-Grasper-C
  8. Stationary Scissor’s Handle.
  9. Movable Scissor’s Handle.
10. Push Lever-A
11. Adaptor-A
12. Push Lever-A2
13. Adaptor-A2
14. Compression Spring A
15. Mini-Rod-A
16. Jaws-A
17. Mini-Sheath-A
18. Push Lever-B
19. Adaptor-B
20. Push Lever-B2
21. Adaptor-B2
22. Compression Spring-B
23. Mini-Sheath -B
24. Mini-Rod B
25. Jaws-B
26. Mini-Sheath-C
27. Mini-Rod-C
28. Ball-C
29. Compression Spring-C
30. Jaws-C
31. Fulcrum Screw-C
33. Fixation Screw-C
34. Union Screws-C. x2
35. Master Screws, x3.
```

### DETAILED DESCRIPTION OF THE DRAWINGS

### Detailed Description of the Drawings of the Instrument

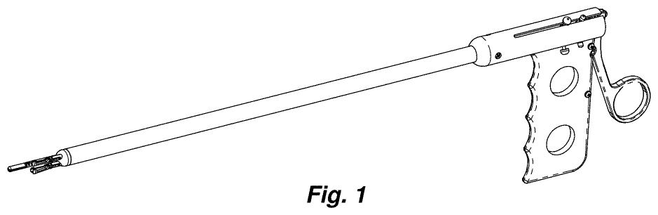

Sheet 1—FIG. 1 shows a perspective view of the left side of the entire instrument.

Sheet 2—FIG. 2 shows the Main Sub-assembly, consisting of the main sheath 1, the front spacer 2, the rear spacer 3, and the chamber with handle 4.

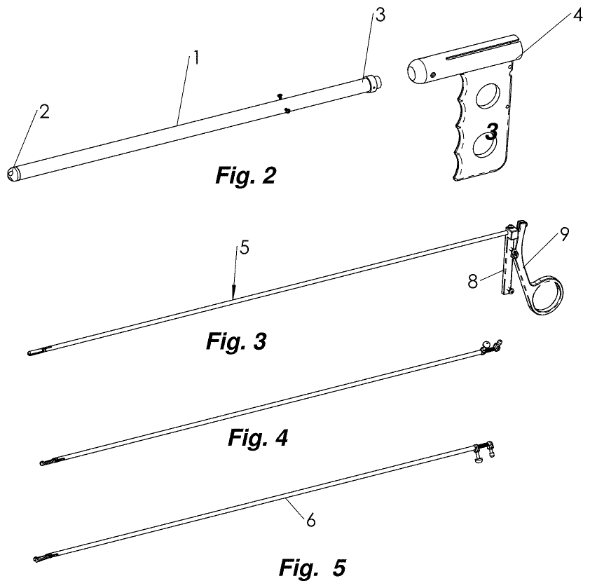

FIG. 3 is a perspective view of the entire stationary mini-grasper, with 5 the mini-sheath-C.

FIG. 4 shows the Slidable Mini-Grasper-A.

FIG. 5 shows the Slidable Mini-Grasper-B, with 6 the mini-sheath-B.

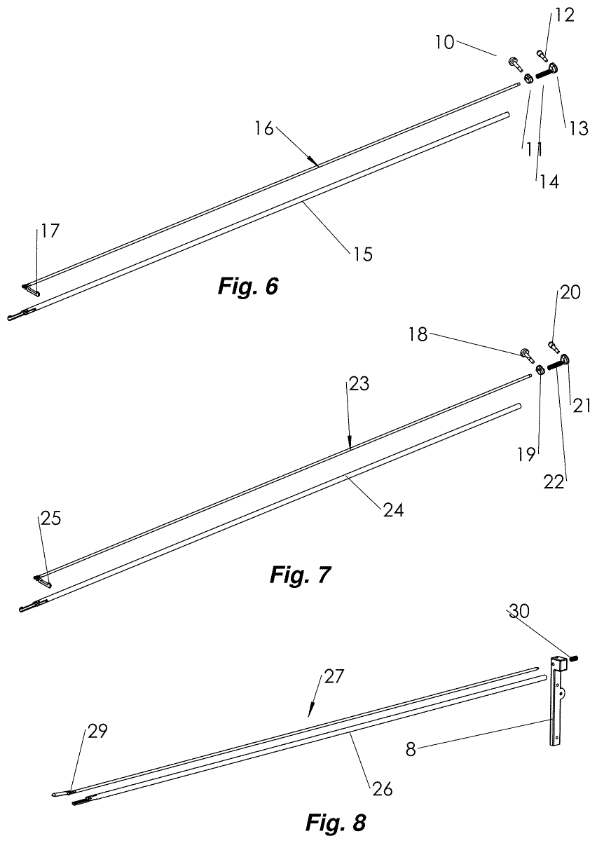

Sheet 3—FIG. 6 shows an exploded view of the Mini-Grasper A, which resides in the upper left side of the main sheath, showing the Push-Lever-A, 10; the Adaptor-A, 11; the Push-Lever-A2, 12; the Adaptor-A2, 13; the compression spring-A, 14; the mini-sheath A, 15; the mini-rod A, 16; the movable jaw-A, 17.

FIG. 7 shows an exploded view of the Mini-Grasper-B, which is identical to Mini-Grasper-A, showing the Push-Lever-B, 18; the Adaptor-B, 19; the Push-Lever-B 2, 19; the Adaptor-B 2, 13; the compression spring-B, 22 ; the mini-sheath B, 26; the mini-rod-B, 27; the movable jaw-B, 29.

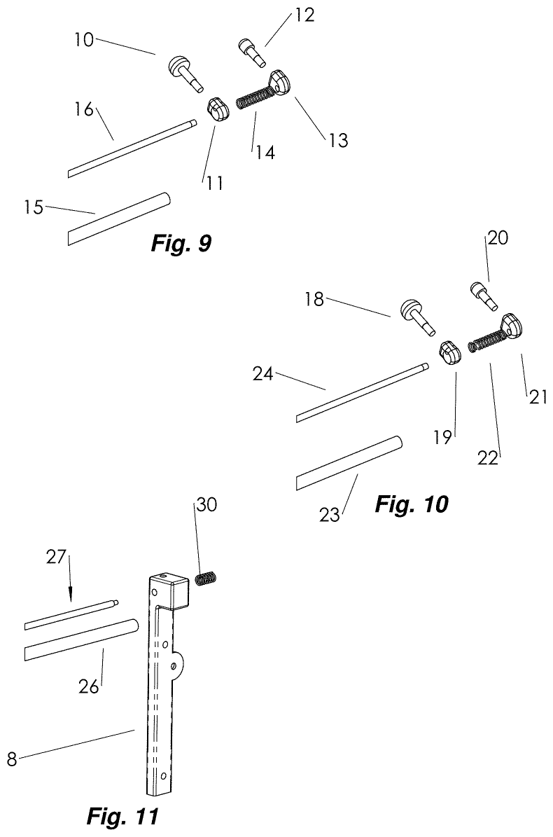

Sheet 4—FIG. 9 shows an exploded view of the rear end of Mini-Grasper A, which is exactly the same as Mini-Grasper B, and resides in the left upper side of the main sheath. The Push-Lever-A, 10; the Adaptor-A, 11; the Push-Lever-A2, 12; the Adaptor-A2, 13; the compression spring-A, 14; the mini-sheath-A, 15; the mini-rod A, 16.

FIG. 10 shows an exploded view of the rear end of Mini-Grasper B, which is exactly the same as Mini-Grasper A, and resides in the left lower side of the main sheath. The Push-Lever-B, 18; the Adaptor-B, 19; the Push-Lever-B2, 20; the Adaptor-B 2, 21; the compression spring-B, 22; the mini-sheath-B, 23; the mini-rod-B, 24.

FIG. 11 shows an exploded view of the Stationary member of the Stationary Grasper-C, 8. which resides on the right side of the main sheath; showing the mini-sheath-C, 26; the mini-rod- C, 27; the compression spring-C, 30.

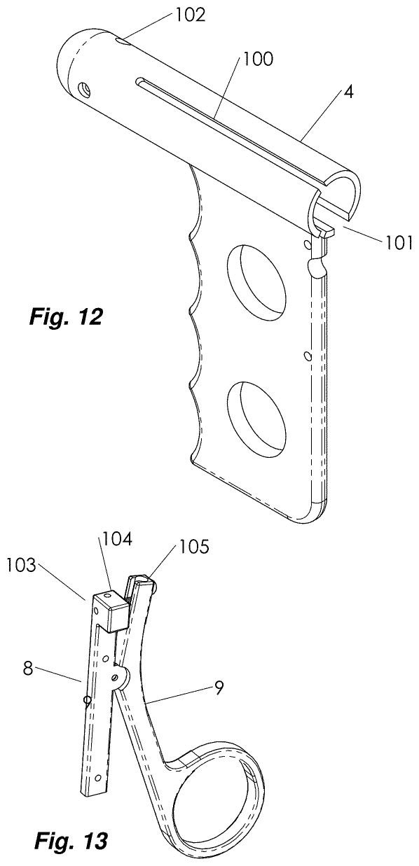

Sheet 5—FIG. 12 shows a perspective view of the Common Tubular Chamber with the Handle, 4, showing the upper long slot of the left side of the chamber, 100; the short slot accommodating the scissors handles, 101; the holes for the Master Screws, 102.

FIG. 13 is a view of both members of the scissors handles; 8 is the stationary member; 9 is the movable handle; 103 is a through hole for passing the mini-sheath C through the vertical member of the scissors handle; 104 is a thread hole the fixation screw; 105 is a half round knob attached to the back surface of the top of the movable handle, enabling pushing by the thumb.

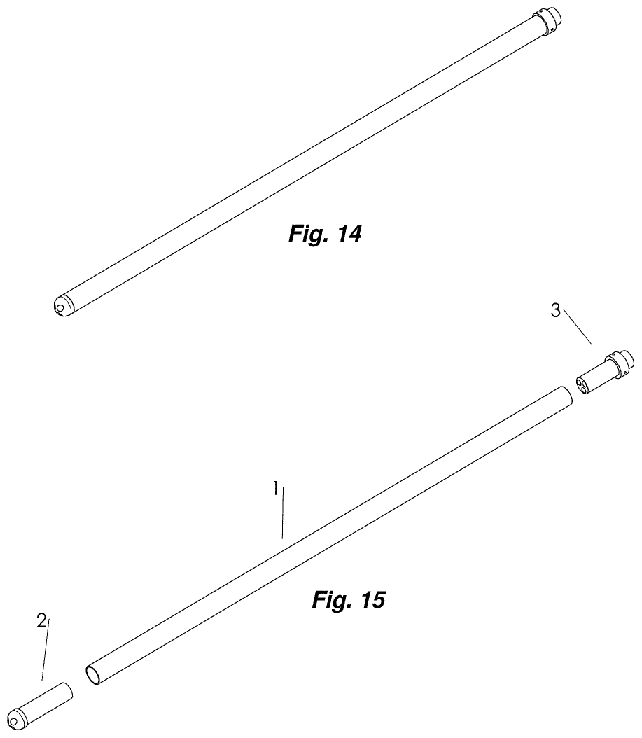

Sheet 6—FIG. 14 shows the Main Sub-assembly.

FIG. 15 shows an exploded view of this, showing the Main Sheath, 1; the Front Spacer, 2; the Rear Spacer, 3.

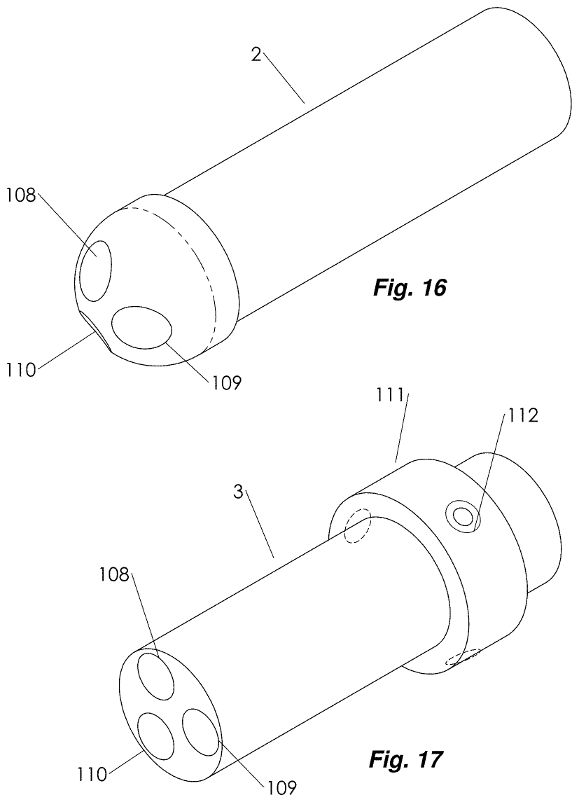

Sheet 7—FIG. 16 shows a perspective of the Front Spacer 2, with 108 being the guide channel for mini-grasper-A; 109 for mini-grasper-B, 110 for mini-grasper-C.

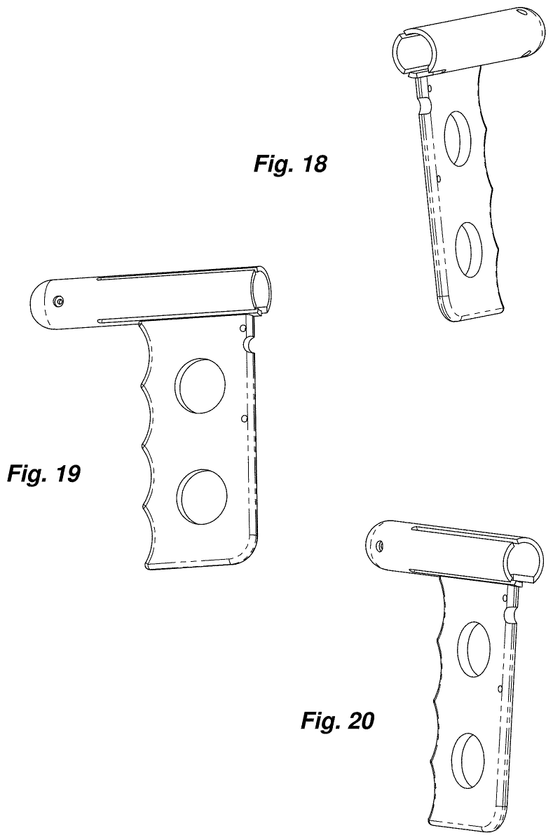

FIG. 17 shows a perspective view of the Rear Spacer 3, which is at the front of the tubular chamber, with 108 being the guide channel for mini-grasper-A; 109 for mini-grasper-B, 110 for mini-grasper-C. 111 is a shoulder on the rear spacer, which abuts the internal shoulder on the front end of the tubular chamber. 112 is the threaded hole for the Master Screws, there being 3 of them, fixing the mini-grasper sub-assembly to the Tubular Chamber.

Sheet 8—FIGS. 18, 19, and 20 show different views of the Chamber-Handle combination.

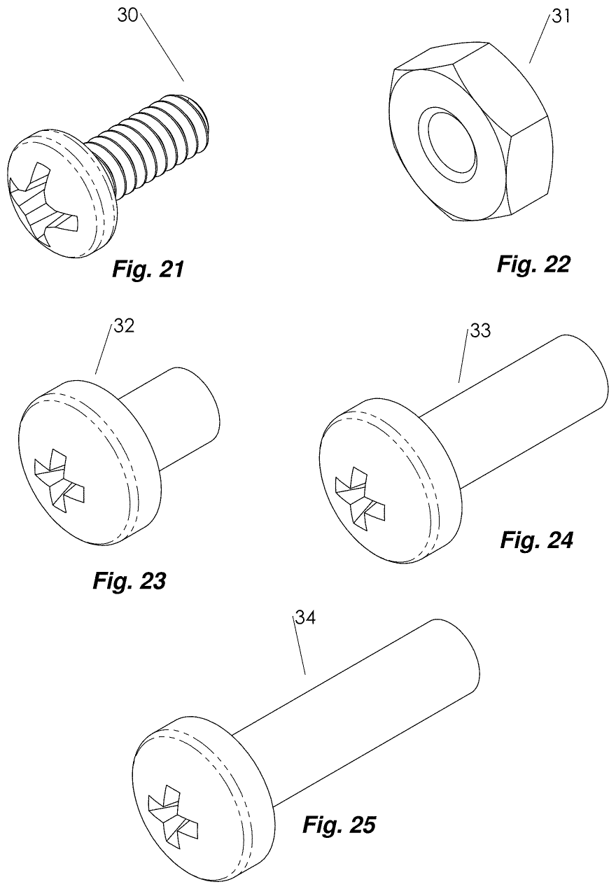

Sheet 9—FIG. 21 shows the Fulcrum Screw for the Scissors Handles.

FIG. 22 shows the Nut for the Fulcrum screw.

FIG. 23 is the Fixation Screw for the top of the stationary member of the scissors handle.

FIG. 24 is the Master Screw, with 3 of them.

FIG. 25 is the Union Screw which binds the stationary member of the scissors handle to the handle of the Chamber.

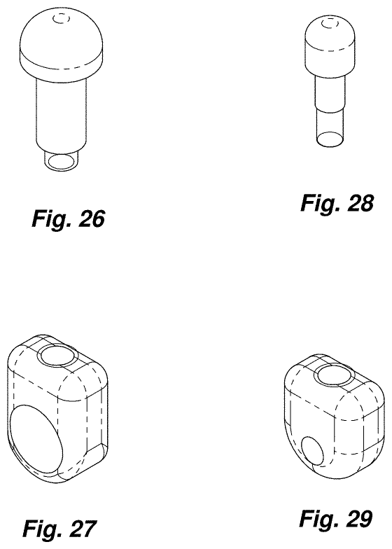

Sheet 10—FIG. 26 shows the Push Lever-A.

FIG. 27 shows the Adaptor-A.

FIG. 28 shows the Push Lever-A2.

FIG. 29 shows the Adaptor-A2. There is an identical set ofthese 4 items for mini-graspers A and B.

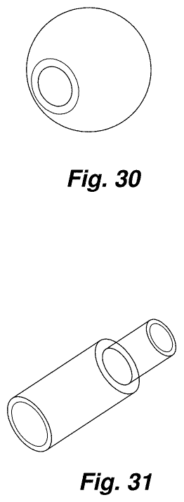

Sheet 11—FIG. 30 shows the Ball at the end of the Rod of the Stationary Grasper C. FIG. 29 shows the male threaded end of the Rod-C for attaching the Ball.

Sheet 12—FIG. 30 shows the jaws of the stationary grasper, grasper-C, in a closed position.

FIG. 31 shows the same jaws in an open position. These are regular jaws of prior art. 113 is the stationary jaw, which is a part of the mini-sheath. 114 is a movable jaw.

Sheet 13—FIG. 32 shows the angled Jaws of a Slidable Grasper, in the closed position.

FIG. 33 shows these jaws in the open position. These jaws are the same for mini-grasper A, and mini-grasper B. 115 is the stationary jaw. 116 is the movable jaw.

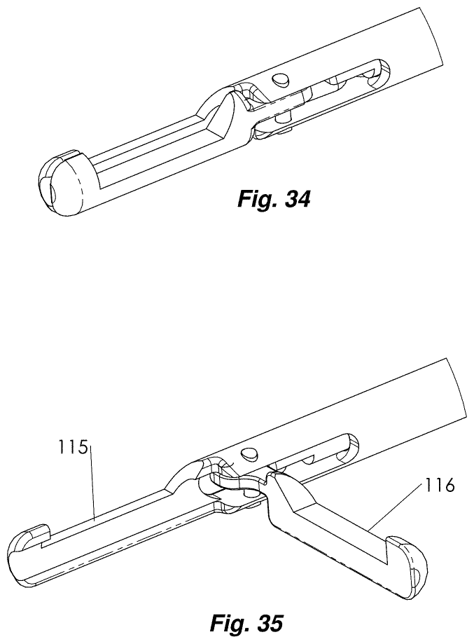

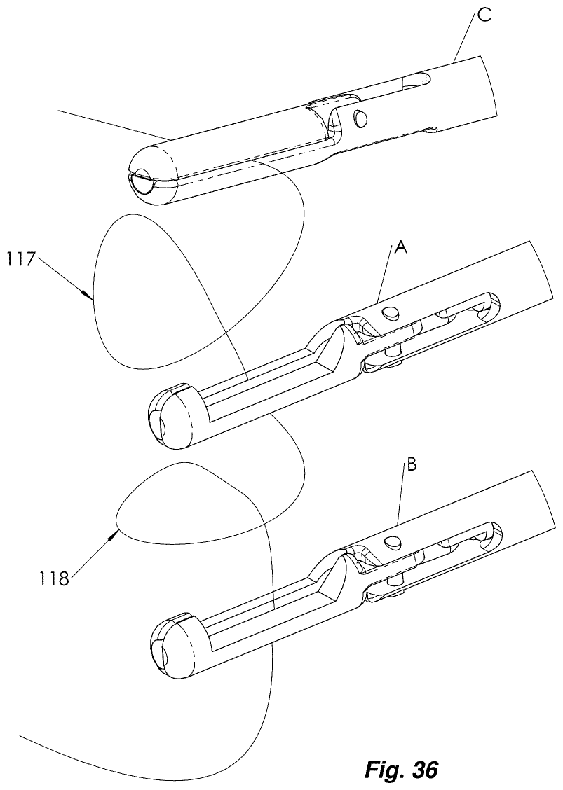

Sheet 14—FIG. 34 shows how the two loops in the suture are made with the 3 mini-graspers, A, B, & C. 117 is the first loop. 118 is the second loop. It is in this situation when the two loops are being held by the mini-graspers, that the independent separate regular grasper D, comes in, goes through the loops, to pull the tail end of the suture back through the loops.

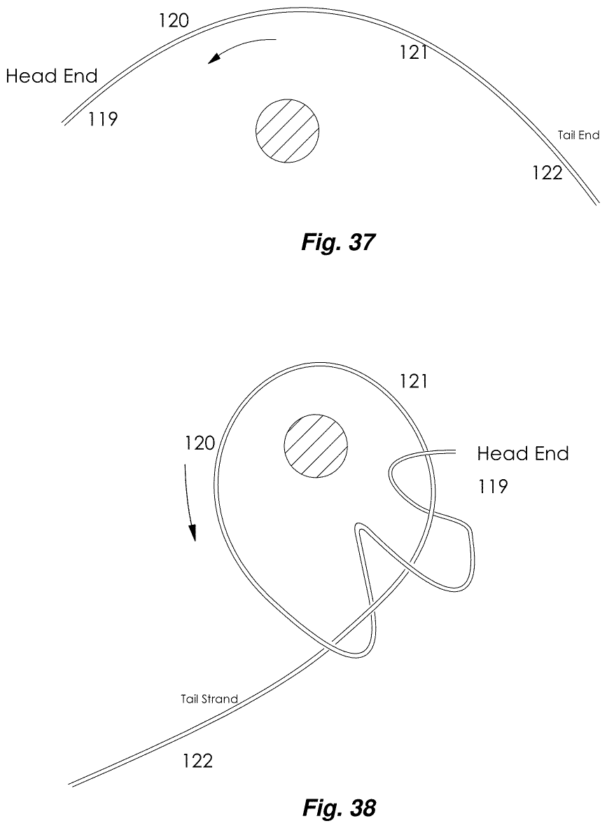

Sheet 15—FIG. 37 is a diagram showing the different parts of a suture, with 119 being the Head End, 120 the Head Strand, 121 the Tail Strand, and 122 the Tail End.

FIG. 38 shows the two loops, with the tail strand pulled through the loops.

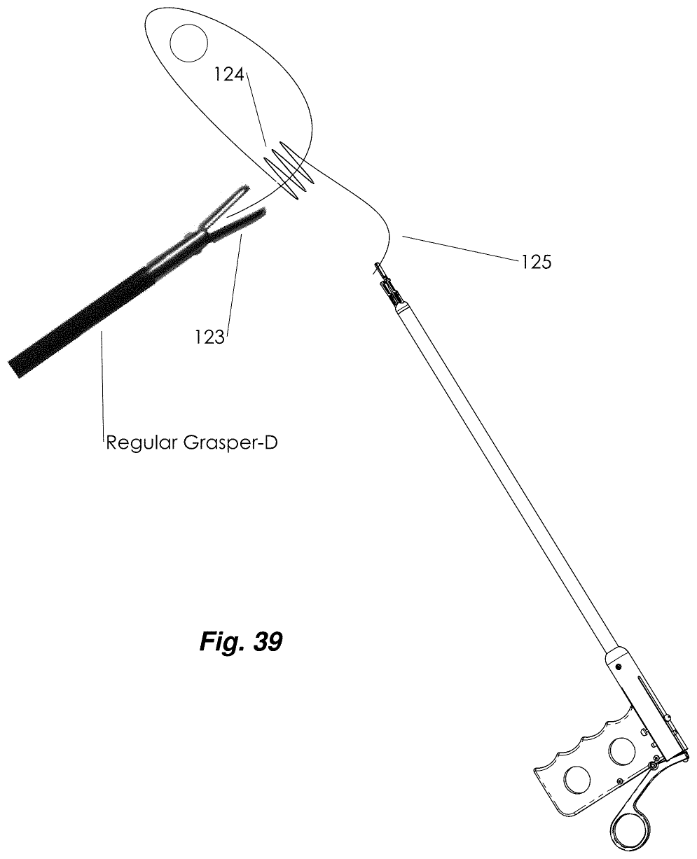

Sheet 16—FIG. 39 shows a separate regular 5 mm grasper, Grasper-D, through a separate port, pulling the Tail End 123 through the two loops, 124, whilst the Head End of the suture, 125, is being held by the mini-grasper-C. This is the reason for mini-grasper-C having a scissors handle, so that the head end of the suture can be firmly gripped and pulled.

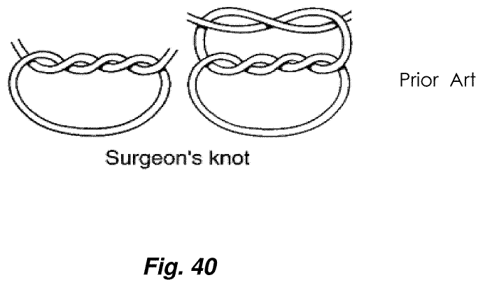

Sheet 17—FIG. 40 shows the Surgeon's Knot, with 2 loops in the first throw.

### Sequence of Assembly

1. First, the main sheath sub-assembly is created by using 3 pieces of 3 mm guide rods, to line up the 3 lumens in the front spacer, and the rear spacer in the correct orientation within the main sheath. The front spacer and the rear spacer are then joined to the main sheath, using adhesives. The guide rods are then removed.
2. The 2 slidable mini-graspers with their jaws, push levers, adaptors and compression springs are assembled.
3. The 2 slidable mini-graspers are inserted in turn into the rear end of the chamber, then through the rear spacer, and out of the front spacer. These mini-graspers are correctly orientated so that their stationary jaws all face the center of the spacers, and their push levers are within their slots.
4. The shaft of the stationary mini-grasper-C is inserted through the lumen at the top end of the stationary member of the scissors, and temporarily locked in position with the fixation screw.
5. The stationary member of the scissors handle is then joined to the movable handle with the fulcrum screw and nut, whilst simultaneously assembling the compression spring-C, and placing the ball into the top of the movable handle.
6. The stationary mini-grasper C is adjusted to achieve the correction orientation.
7. The shoulder on the rear spacer is then mated to the internal shoulder in the neck of the tubular chamber, with 3 counter-sunken master-screws.
8. The stationary member of the scissors handle is joined to the handle portion of the chamber, with 2 union-screws.

1. An instrument for laparoscopically making multiple loops in a suture and for tying a surgeon's knot, comprising of two tubular sheaths, an inner sheath inside a main sheath, separated by adaptors at each end, with the lumen of the inner sheath open at both ends and containing the shaft of a removable 5 mm laparoscopic grasper, acting as the axis of rotation, with the said grasper and the said instrument being used together in the same laparoscopic port, with the main sheath further bearing a controllable mechanism capable of attaching or releasing a tail end of the suture at its distal end.

2. A method for laparoscopically making multiple loops in a suture and for tying a surgeon's knot with the instrument of claim 1, comprising the following steps:
a) The tail end of a suture is first attached to the distal surface of the main sheath;
b) The main sheath is repeatedly rotated, forming multiple loops in the suture surrounding the main sheath;
c) These loops are displaced to lie on the exposed portion of the shaft of a 5 mm laparoscopic grasper contained within the central lumen of the inner sheath;
d) The head end of the said suture is pulled through the said loops, by the said 5 mm grasper already in place within the said loops;
e) The tail end of the suture is grasped by a second grasper;
f) The two ends of the suture are pulled apart from each other, thus forming and tightening a surgeon's knot.
